(19) United States
(12) Patent Application Publication
(10) Pub. No.: US 2022/0023521 A1
(43) Pub. Date: Jan. 27, 2022

(54) TECHNIQUES FOR COLLECTING DIALYSIS EFFLUENT TARGET MATERIALS

(71) Applicant: Fresenius Medical Care Holdings, Inc., Waltham, MA (US)

(72) Inventors: Mohamad Imtiaz Hakim, New York, NY (US); Xia Tao, New York, NY (US); Mia Garbaccio, New York, NY (US); Peter Kotanko, New York, NY (US); Xiaoling Wang, New York, NY (US)

(21) Appl. No.: 17/381,594

(22) Filed: Jul. 21, 2021

Related U.S. Application Data
(60) Provisional application No. 63/056,078, filed on Jul. 24, 2020.

(51) Int. Cl. A61M 1/28

(52) U.S. Cl. CPC A61M 1/285; A61M 2202/09; A61M 1/282

(57) ABSTRACT

Techniques and devices for filtering dialysis fluids, such as dialysis effluent, are described. For example, a filter device may be configured to filter peritoneal dialysis (PD) effluent draining from a patient during a PD process. The filter devices may include filters configured to filter materials, such as human cells, microorganisms, and/or other components from PD effluent. For instance, a filter device may be configured to be installed in-line in a drain circuit of a PD system using conventional tubing. The captured materials may be analyzed or otherwise processed to determine health characteristics of a patient and/or to capture stem cells. Other embodiments are described.

### CROSS-REFERENCE TO RELATED APPLICATION

This application claims the benefit of priority of 35 U.S.C. § 119(e) to U.S. Provisional Patent Application Ser. No. 63/056,078, filed on Jul. 24, 2020, which is incorporated by reference in its entirety as if fully set forth herein.

### FIELD

The disclosure generally relates to determining physical characteristics of dialysis patients, and more particularly to processes for collecting target materials, such as cells, microorganisms, and/or the like, from peritoneal dialysis (PD) patients during a PD treatment.

### BACKGROUND

In general, a peritoneal dialysis (PD) treatment includes the steps of filling the peritoneal cavity of the abdomen with dialysate, allowing the dialysate to dwell in the peritoneal cavity for a predetermined period, then draining the PD effluent (or spent dialysate) from the peritoneal cavity. Patient treatment success in PD is dependent on monitoring overall patient health, including the presence of infections and the functional and morphological integrity of the peritoneal membrane. In general, the peritoneal membrane is a key component for successful PD treatment, particularly for end stage renal disease patients. For example, morphological changes of mesothelial cells may be associated with the progress of peritoneal membrane remodeling in PD populations. The PD effluent may include materials, such as patient cells, microorganisms, and other elements that may be useful for various purposes.

Conventional PD systems either do not collect any material from PD effluent or rely on inefficient techniques, for instance, that ultimately requiring costly and time-consuming post-processing techniques to gain insight from the collected material. Accordingly, PD patients and healthcare providers would benefit from processes and devices capable of efficiently and effectively collecting target materials from PD effluent using a device that may be employed using existing PD components.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In one aspect of the present disclosure, a collection-valve filter device for filtering dialysis effluent may include a filter body comprising an inlet port, an outlet port, and a collection port, a catheter valve, a collection valve, and a filter arranged between the inlet port and the outlet port to filter the dialysate effluent.

In one aspect of the present disclosure, a collection-valve filter device for filtering dialysis effluent may include a filter body comprising an inlet port, an outlet port, and a collection port, the inlet port and the outlet port arranged at opposite ends of the filter body, the collection port arranged in a bottom portion of the filter body, the inlet port configured to receive the dialysis effluent draining from a catheter fluidically coupled to a patient, a catheter valve configured to move to one of an inlet closed position to close the inlet port or an inlet open position to open the inlet port, and a collection valve operably coupled to the collection port, the collection valve to move to a collection open position to open the collection port and a collection closed position to close the collection port.

In some embodiments of the collection-valve filter device, the collection-valve filter device may be arranged in a drain configuration responsive to the catheter valve being in the inlet open position and the collection valve being in the collection closed position. In various embodiments of the collection-valve filter device, the collection-valve filter device may be arranged in a collection configuration responsive to the catheter valve being in the inlet closed position and the collection valve being in the collection open position. In some embodiments of the collection-valve filter device, the collection-valve filter device may be operative to allow a flow of the dialysis effluent through the inlet port and the filter, and filtrate out through the outlet port, in the drain configuration. In some embodiments of the collection-valve filter device, the collection-valve filter device may be operative to allow a flow of fresh dialysate through the outlet port, the filter, and out through the collection port in the collection configuration. In various embodiments of the collection-valve filter device, the filter may operate to capture materials in the dialysis effluent, the materials comprising at least one of patient cells or microorganisms. In exemplary embodiments of the collection-valve filter device, the collection-valve filter may be configured to filter peritoneal dialysis (PD) effluent during a PD treatment. In various embodiments of the collection-valve filter device, the collection-valve filter may be configured to filter peritoneal dialysis (PD) effluent during continuous ambulatory peritoneal dialysis (CAPD) treatment. In some embodiments of the collection-valve filter device, the collection-valve filter device may be configured to be installed in drain tubing of a continuous ambulatory peritoneal dialysis (CAPD) system.

In one aspect of the present disclosure, a method of treating a dialysis patient may include collecting a peritoneal dialysis (PD) effluent residue using a collection-valve filter device operating according to some embodiments, analyzing the residue to determine a health condition of the dialysis patient, and determining a treatment recommendation based on the health condition.

In one aspect of the present disclosure, a method of treating a dialysis patient may include collecting a peritoneal dialysis (PD) effluent residue during a PD treatment of the patient using a collection-valve filter device operatively coupled to a PD system, the collection-valve filter device may include a filter body, an inlet port, an outlet port, and a filter arranged between the inlet port and the outlet port to filter the dialysis effluent and collect the PD effluent residue. The method may further include analyzing the PD effluent residue to determine a health condition of the dialysis patient and determining a treatment recommendation based on the health condition

In some embodiments of the method, the treatment recommendation may include harvesting stem cells from the residue. Various embodiments of the method may include administering the treatment recommendation.

In one aspect of the present disclosure, a container filter device for filtering dialysis effluent may include a filter body, an inlet port, an outlet port, and a filter arranged between the inlet port and the outlet port to filter the dialysis effluent.

In one aspect of the present disclosure, a container filter device for filtering dialysis effluent may include a filter body having an inlet port and an outlet port arranged at opposite ends thereof and a filter arranged between the inlet port and the outlet port, wherein, in a drain configuration, the inlet port may be operative to receive the dialysis effluent from a drain line of a dialysis system, the dialysis effluent may flow through the filter to capture residue in the dialysis effluent and generate filtrate to flow out of the container filter device via the outlet port.

In some embodiments of the container filter device, the inlet port may be closed and a fluid container is fluidically coupled to the outlet port when the container filter device is in a preservation configuration. In various embodiments of the container filter device, the outlet port is configured to receive fluid arranged within the fluid container, the fluid to flow through the filter into the filter body, when the container filter device is in a preservation configuration. In some embodiments of the container filter device, the filter may be configured to capture materials in the dialysis effluent, the materials may include at least one of patient cells or microorganisms. In various embodiments of the container filter device, the collection-valve filter may be configured to filter peritoneal dialysis (PD) effluent during a PD treatment. In exemplary embodiments of the container filter device, the collection-valve filter may be configured to filter peritoneal dialysis (PD) effluent during automated peritoneal dialysis (APD) or continuous cyclic peritoneal dialysis (CCPD) treatment. In some embodiments of the container filter device, the collection-valve filter device may be configured to be installed in drain tubing of an automated peritoneal dialysis (APD) or a continuous cyclic peritoneal dialysis (CCPD) system.

In one aspect of the present disclosure, a method of treating a dialysis patient may include collecting a peritoneal dialysis (PD) effluent residue using a container filter device according various embodiments, analyzing the residue to determine a health condition of the dialysis patient, and determining a treatment recommendation based on the health condition. In some embodiments of the method, the treatment recommendation may include harvesting stem cells from the residue. In various embodiments of the method, may include administering the treatment recommendation.

### BRIEF DESCRIPTION OF THE DRAWINGS

By way of example, specific embodiments of the disclosed machine will now be described, with reference to the accompanying drawings, in which:

FIG. 1 illustrates a first exemplary operating environment according to some embodiments;

FIG. 2A illustrates a first collection-valve filter device according to some embodiments;

FIG. 2B illustrates the collection-valve filter device of FIG. 2A in a drain configuration according to some embodiments;

FIG. 2C illustrates the collection-valve filter device of FIG. 2A in a collection configuration according to some embodiments;

FIG. 3 depicts a continuous ambulatory peritoneal dialysis (CAPD) configuration according to some embodiments;

FIG. 4A illustrates a second collection-valve filter device in a drain configuration according to some embodiments;

FIG. 4B illustrates the second collection-valve filter device in a collection configuration according to some embodiments;

FIGS. 5A-5G depict various views of components of a collection-valve filter device according to some embodiments;

FIG. 6 illustrates a method flow for collecting PD effluent material using a collection-valve filter device according to some embodiments;

FIG. 7A illustrates a first container filter device in a drain configuration according to some embodiments;

FIG. 7B illustrates the container filter device of FIG. 7A in a preservation configuration according to some embodiments;

FIG. 7C illustrates the collection-valve filter device of FIG. 7A in a container configuration according to some embodiments;

FIG. 8 depicts an automated peritoneal dialysis (APD) or continuous cyclic peritoneal dialysis (CCPD) configuration according to some embodiments;

FIG. 9A illustrates a second container filter device in a drain configuration according to some embodiments;

FIG. 9B illustrates the second container filter device of FIG. 9A in a preservation configuration according to some embodiments;

FIG. 10 depicts various views of a container filter device according to some embodiments;

FIG. 11 illustrates a method flow for collecting PD effluent material using a container filter device according to some embodiments; and

FIGS. 12A and 12B illustrate an exemplary PD system according to some embodiments.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

Patient treatment success in peritoneal dialysis (PD) is dependent on the functional and morphological integrity of the peritoneal membrane. In addition to functional failure of the peritoneum, long-term PD may lead to anatomical changes in the peritoneal tissues such as neoangiogenesis, vasculopathy and fibrosis, sometimes causing peritoneal sclerosis. Membrane characteristics alter especially after sustained use of non-physiological dialysis fluids. Accordingly, patient characteristics may be monitored over the duration of a PD patient treatment regimen to ensure, among other things, the health of patient peritoneal anatomy and/or the effectiveness of PD treatment. Non-limiting patient characteristics may include peritoneal transport status, dialysis adequacy, membrane characteristics, unexplained clinical changes, ultrafiltration failure, and/or the like.

The PD effluent may include materials, such as patient cells, microorganisms, and other elements that may be useful for various purposes. For example, examinations of the molecular and morphological changes of exfoliative cells from PD effluent can reveal information for diagnostic purpose. Mesenchymal stem cells (MSCs) and hematopoietic stem cells (HSCs) may be found in PD effluent. MSCs and HSCs are a potential therapeutic agent to repair the degraded membrane, reduce fibrosis, reduce inflammation, and other ailments associated with peritoneal dialysis complications. A collection of patient MSCs may be stored and expanded in a lab, for example, as stem cell bank for future therapeutic uses in PD patients. In a further example, during infection episodes, such as peritonitis, there could be detectable microorganisms present in PD effluent. The detection of these microorganisms, especially their gram species, using conventional collection and/or filtration systems must rely on a laboratory examination after culture enrichment.

Conventional PD treatment systems typically do not provide devices for the collection of PD effluent and/or materials contained within PD effluent in a manner that can be analyzed by laboratory or healthcare professionals. In contrast, a typical PD system provides a patient with a waste container used to collect the PD effluent for disposal purposes only. Specialized collection containers may be used to collect PD effluent that may be provided to a laboratory for analysis. However, the collection containers must be used by a patient in a clinical facility and/or require costly and time-consuming processing by the laboratory in order to obtain materials of interest (for instance, cells, microorganisms, and/or the like) in the PD effluent. Standard devices for filtering bodily fluids have generally proven to be ineffective for PD effluent and are not specialized for the unique configuration of PD systems.

Accordingly, some embodiments may provide PD effluent filter devices that may be used to collect material of interest from PD effluent during a PD process. Non-limiting examples of materials of interest may include cells, bacteria, fungi, and/or other materials. A PD effluent filter according to some embodiments may be used for various PD processes, including, without limitation, continuous ambulatory peritoneal dialysis (CAPD), automated peritoneal dialysis (APD), continuous cyclic peritoneal dialysis (CCPD), and/or the like. A PD effluent filter device may be used, for example, to capture and preserve cells, microorganisms, and other materials that are eluted into peritoneal dialysis (PD) effluent during routine PD fluid exchange. The captured materials may be collected for diagnostic, therapeutic, or other purposes. A PD effluent filter may be configured as an aseptic device designed, for instance, to be single use as needed in both APD and CAPD patients.

PD effluent filters according to some embodiments may provide multiple technological advantages over existing systems. For example, in one non-limiting technological advantage, PD effluent filters may be patient-friendly and may be used with existing PD systems, for example, with conventional tubing sets. In this manner, PD effluent filter devices according to some embodiments may be used by a patient in their own home during a dialysis treatment. In another example non-limiting technological advantage, PD effluent filter devices may provide for the concentrating of cells, microorganisms, and/or other materials collected on a filter system that may facilitate the detection of these materials by a point-of-care device without extensive processing such as culture enrichment.

Although PD processes and filter devices for PD patients are described in some examples, embodiments are not limited to PD configurations. For example, filter devices described according to some embodiments may be used in fluid circuits for other types of dialysis or other filtering processes. Embodiments may not be limited in this context.

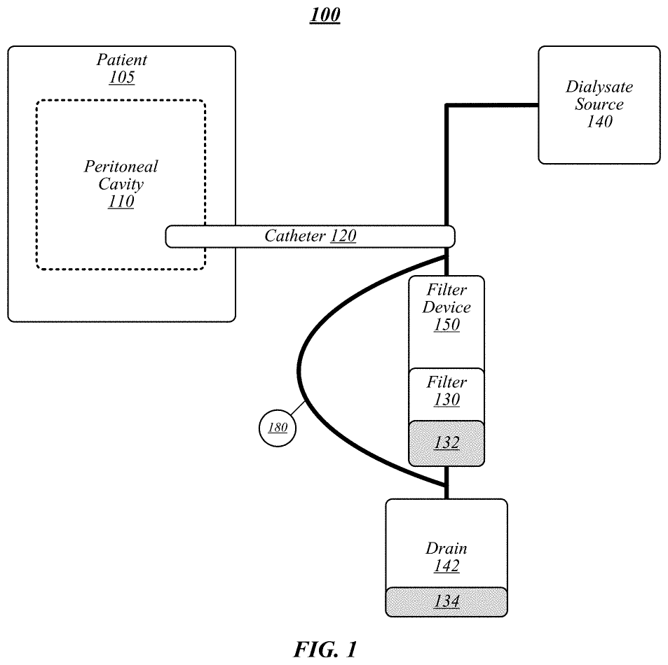

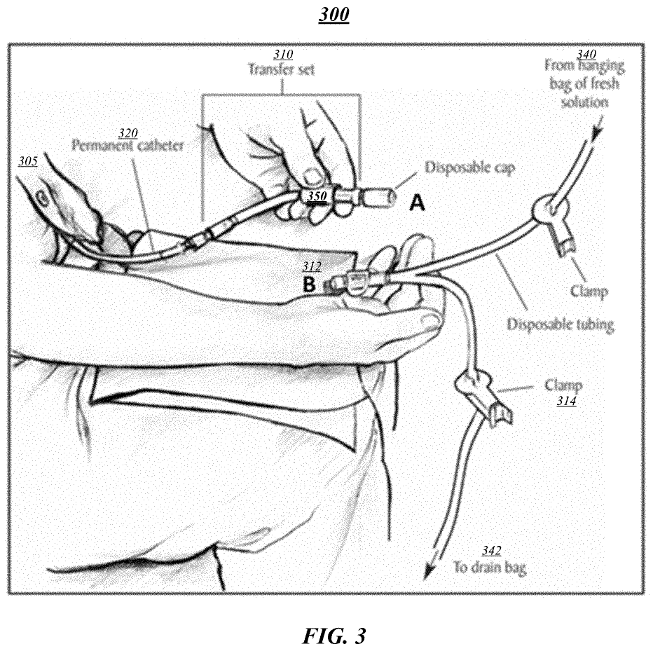

FIG. 1 illustrates an example of an operating environment 100 that may be representative of some embodiments. In general, operating environment 100 may include a configuration for performing a PD process according to some embodiments. As shown in FIG. 1, a patient 105 may have a catheter 120 arranged within a peritoneal cavity. A dialysate source 140 may provide dialysate that may flow through catheter 120 into peritoneal cavity 110. In some embodiments, dialysate source 140 may be a part of a CAPD system (see, for example, FIG. 3) or an APD system (see, for example, FIG. 8). In either a CAPD system or an APD system, PD effluent may exit peritoneal cavity 110 via catheter 120 and flow to drain 142. In various embodiments, a filter device 150 may be arranged between catheter 120 and drain 150 to filter the PD effluent. Filter device 150 may include a filter 130 configured to capture a residue 132 (for instance, materials removed from PD effluent via filter 130) from the PD effluent. Filtrate 134 (for instance, PD effluent with residue 132 removed via filter 130) may flow to drain 142.

Filter 130 may be formed of various filter materials. For example, filter 130 may be or may include a mesh or membrane filter made of polyethylene terephthalate (PET), polytetrafluoroethylene (PTFE), mixed cellulose esters (MCE), cellulose acetate, polycarbonate, nylon, polymer materials, fibrous materials, metal materials, variations thereof, combinations thereof, and/or the like. In some embodiments, the materials and/or characteristics of filter 130 (for instance, pore size) may be determined based on target applications. For example, to collect bacteria, a filter material with size below 0.45 μm may be used; to collect fungi, a 0.65 μm filter will be used; to collect human cells, a 5 μm or larger size filter may be used. In various embodiments, the pore size may be about 1 μm, about 5 μm, about 10 μm, about 20 μm, about 40 μm, about 45 μm, about 50 μm, about 65 μm, about 100 μm, about 500 μm, and any value or range between any two of these values (including endpoints). In various embodiments, filter materials may be or may include chemically or biologically modified membranes or materials to target (or, alternatively, repel) certain materials. For example, an antibody coated membrane may be used to capture white blood cells and bacteria, for example, selectively for diagnostic purposes.

In various embodiments, filtration of PD effluent via filter device 150 may be through a gravity-based flow during draining of PD effluent. In various configurations, such as in a configuration using a small pore size filter material, gravity may not provide sufficient force to draw the fluid out from the peritoneal cavity. In such configurations, an additional force may be used to force the PD effluent through filter 130, such as a vacuum pump unit (or additional vacuum pump unit) that may be applied to filter device 150 and before the drain (for example, a drain bag tubing set in a CAPD configuration) to create pressure to facilitate PD effluent drain out from peritoneal cavity. In various embodiments, efficient draining flow may be facilitated using acoustic wave separation technology, for instance, coupled with a large pore size filter material. When large pore size filter is used, gravity (or the conventional pump system of an APD configuration) may provide sufficient force to drain out PD effluent, however cells or microorganisms may not be efficiently captured because the pore size could be too big to retain them. Accordingly, some embodiments may use an acoustic wave device applied at the filter location, for instance, to generate a 3-dimensional standing wave across the filter by use of low frequency acoustic forces. Cells, microorganisms, and/or other target materials may be trapped by the acoustic forces and retained on the filter membrane. Non-limiting examples of acoustic wave devices may include a surface acoustic wave (SAW) device, piezoelectric devices, ultrasonic devices, and/or the like.

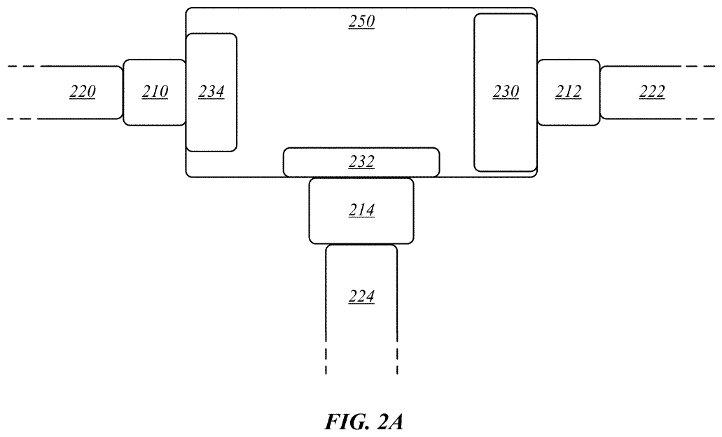

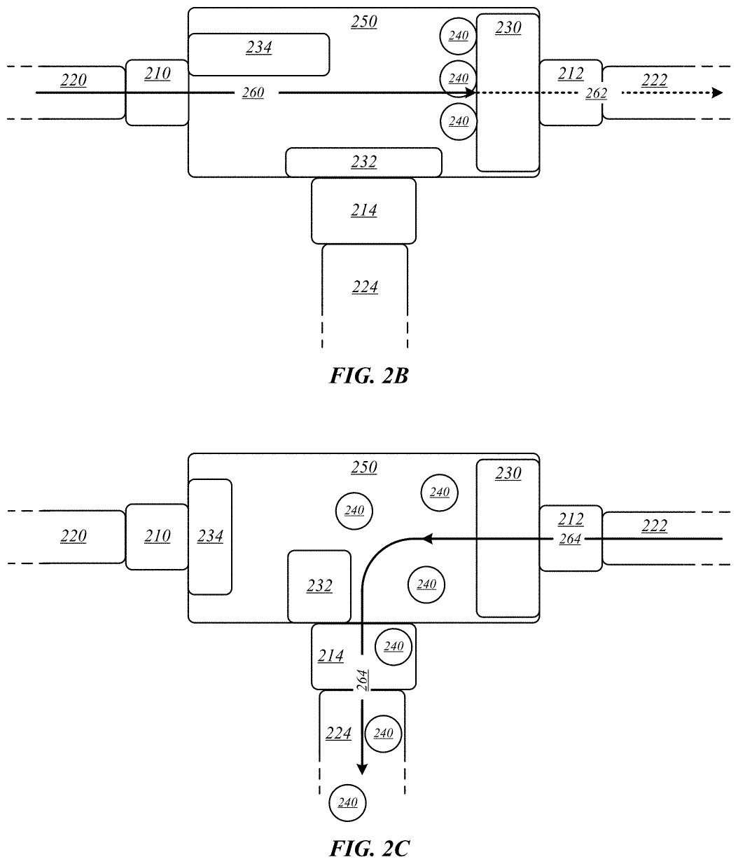

Filter device 150 may include various configurations according to some embodiments. For example, filter device 150 may be configured as a collection-valve filter device (see, for example, FIGS. 2A-2C and 4A-5G). In general, for a collection-valve filter device configuration, filter device 150 may be placed in a drain configuration in which a drain valve is open and a collection valve is closed. During the PD draining process, PDF effluent may flow through the drain valve of filter device 150, through filter 130, and out through an outlet port. During draining, residue 132 may be captured via filter 130. To collect residue 132, filter device 150 may be placed in a collection configuration in which the drain valve is closed and the collection valve is opened. Fresh dialysate (or another fluid) may flow through the outlet port into filter device (for instance, in the opposite direction of PD effluent flow during PD draining), removing at least a portion of residue 132 from filter and out of filter device via the collection valve. In some embodiments, a collection-valve filtration device may be used for a CAPD process.

For a container filter configuration, filter device 150 may have a drain configuration and a preservation configuration. In the drain configuration, the PD effluent may flow into filter device 150 via an inlet port and through filter 130, and the filtrate of the PD effluent may flow out of filter device 150 through an outlet port. Residue 132 may remain in filter device 150. In the preservation configuration, the inlet port is closed and a fluid may flow into filter device 150 via the outlet port. In some embodiments, the fluid may include a preservation fluid, such as a cell preservation medium (for instance, Dulbecco's Modified Eagle Medium), a microorganism preservation medium (for instance, a nutrient broth for bacteria), and/or the like. In other embodiments, the fluid may be or may include fresh dialysate. In various embodiments, the preservation fluid may be provided in a pouch or other container coupled to the outlet port, for example, that may be squeezed to flow the fluid contents into filter device 150. The outlet port may be sealed (water-tight, hermetically, and/or the like), for example, via a cap or by the container coupled to the outlet. In some embodiments, a container filter device may be used for an APD configuration.

Although in some examples, the container filter device has been indicated for use with an APD configuration and the collection-valve filter device has been indicated for use with a CAPD configuration, embodiments are not so limited because the container filter device and/or the collection-valve filter device may be used with any dialysis configuration capable of operating according to some embodiments.

In various embodiments, a bypass system 180 may be included to allow fluid to bypass filter device 150. For example, if filter device 150 becomes blocked, clogged, or otherwise does not allow a sufficient amount of fluid to flow through filter device 150, bypass system 180 may provide tubing, conduit, valves, sensors, and/or other elements to allow fluid to flow around filter device 150 to drain 142. In one embodiment, a bypass valve or other structure (not shown) may be located between catheter and filter device that may be closed to cause fluid to flow through bypass system 180 and bypass filter device 150. In some embodiments, the bypass valve may be actuated by patient 105 or other user.

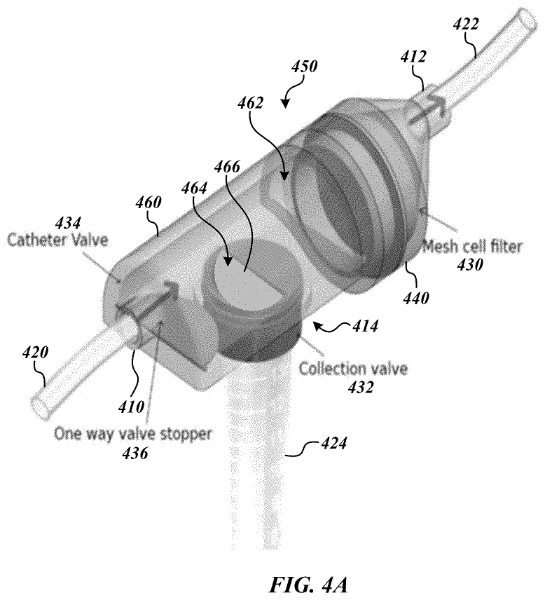

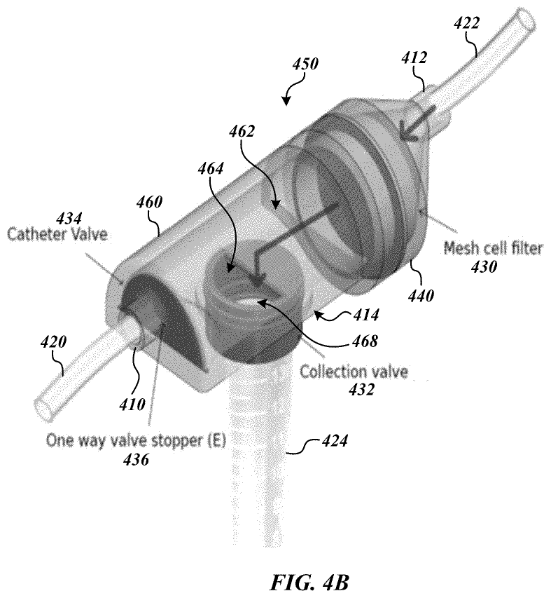

In various embodiments, bypass valve may be actuated automatically, for example, based on a sensor measurement (for instance, indicating a blockage). For example, a pressure sensor reading indicating a buildup of pressure (e.g., high differential pressure) across filter device 150 over a threshold amount and/or a flow meter indicating a level of flow below a predetermined level through filter device 150. In some embodiments, bypass valve may be actuated automatically due to fluid flow that may occur during a blockage, for instance, if fluid cannot flow through filter 130 and the fluid backs up as a result, the backwards flow may close a bypass valve (or other valve, such as a catheter valve; see, for example, FIG. 4A) that may lead to fluid flowing through the bypass circuit. Additional valves or other structures (not shown) may be used in combination with a bypass valve or other structures to facilitate the flow of fluid through bypass system 180. For example, a backflow preventer valve may be positioned (for instance, between catheter and the intersection of bypass system 180 tubing with drain tubing, upstream of filter device 150) to cause any “backwards” fluid flow from filter device 150 to move through bypass system 180. Embodiments are not limited in this context.

FIG. 2A illustrates a collection-valve filter device according to some embodiments. As shown in FIG. 2A, a collection-valve filter device 250 may include an inlet port 210 coupled to tubing 220 in fluid communication with a patient catheter. A catheter valve 234 may be configured to move between an open state and a closed state. In the open state, fluid may flow into filter device 250 via inlet port 210. In the closed state, fluid is prevented from flowing into filter device 250 via inlet port 210. A filter 230 may be arranged within filter device 250 in fluid communication with outlet port 212 coupled to tubing 222. Filter device 250 may include a collection valve 232 in fluid communication with collection port 214 coupled to tubing 224.

FIG. 2B illustrates collection-valve filter device 250 of FIG. 2A in a drain configuration according to some embodiments. In some embodiments, filter device 250 may be in the drain configuration during a draining stage of a PD treatment. As shown in FIG. 2B, catheter valve 234 is in an open state, such that PD effluent 260 may flow into filter device 250 via inlet port 210. PDF effluent 260 may flow through filter 230, leaving residue 240 behind as filtrate 262 exits filter device 250 via outlet port 212. In the drain configuration, no or substantially no fluid passes through collection valve 232. For example, in some embodiments, collection valve 232 may include a valve, door, shelf, protrusion, or other element configured to close or partially close fluid access to collection port. In some embodiments, for instance, collection valve 232 may include or may be operably coupled to an actuator, switch, toggle, pin, release, latch, gear, and/or the like configured to allow a patient or other individual to open and close collection valve 232.

FIG. 2C illustrates collection-valve filter device 250 of FIG. 2A in a collection configuration according to some embodiments. In some embodiments, collection-valve filter device 250 may be placed in the collection configuration after the PD draining stage has completed. As shown in FIG. 2C, in a collection configuration, catheter valve 234 may be in a closed state, preventing the flow of fluid into and out of filter device 250 via inlet port 210. A fluid 264, such as fresh dialysate, a preserving fluid, water, and/or the like, may be flowed into filter device 250 via outlet port 212. In this manner, at least a portion of residue 240 may washed out of filter device 250 and flow through collection outlet 214 to a collection container or other device. In some embodiments, the flow of fluid 264 through filter 230 may operate to remove at least a portion of residue 240 affixed to filter 230.

FIG. 3 depicts a CAPD configuration according to some embodiments. As shown in FIG. 3, a CAPD configuration 300 may include various tubing, conduits, connectors, and other elements to facilitate the proper flow of dialysate into patient 305 in a filling stage and out of patient 305 in a draining stage. Patient 305 may have a permanent catheter 320 in fluid communication with their peritoneal cavity. During CAPD, when patient 305 is ready to drain PD fluid after a dwell, a collection-valve filter device 350 may be attached to their transfer tubing set (A) 310 (for instance, at an inlet end). Drain bag tubing set (B) 312 may be fluidically connected to filter device 350 on the other end (for instance, an outlet end).

Patient 305 may start draining the PD effluent from their peritoneal cavity. Materials in the PD effluent may flow through the filter of filter device 350. For example, cells and microorganisms in the spent dialysate may flow through a mesh filter inside filter device 350 and be captured on the inner surface of the mesh filter. In some embodiments, the catheter valve of filter device 350 may include a one-way catheter valve or door that may open by the force of spent dialysate flow into filter device 350 through the inlet end. In some embodiments, a collection valve or opening between filter device and the collection tube may be closed during the filtering stage (for instance, when filter device 350 is in the drain configuration).

Once all or substantially all of the PD effluent has drained, patient 305 may open the collection valve. Then patient 305 may switch a PD tubing connector to the direction allowing fresh dialysate 330 to flow into filter device 350. The catheter valve may include a door, flap, stopper or other element configured to prevent the catheter valve opening toward the catheter direction. A collection tube, bag, or other container may be fluidically coupled to collection valve to collect the fresh dialysate and residue being rinsed from filter device 350. After a rinsing volume of fresh dialysate has flowed through the mesh filter, infusion of dialysate may be stopped by patient. In some embodiments, the rinsing volume may be about 10 ml. In various embodiments, the rinsing volume may be about 5 ml, about 10 ml, about 20 ml, about 50 ml, about 100 ml, about 1 liter, and any values or ranges between any two of these values (including endpoints). The collection container may be detached from filter device after collection valve has been closed. In some embodiments, the collection container may be stored and/or sent to laboratory for diagnosis, banking services, and/or the like.

FIG. 4A illustrates a collection-valve filter device in a drain configuration according to some embodiments. As shown in FIG. 4A, a filter device 450 may include a housing 460 (transparent in FIG. 4A) having a filter 430 arranged within a cavity 462 defined by housing 460. Filter device 450 may include an inlet port 410 coupled to drain tubing 420. Inlet port 410 may be in fluid communication with a catheter valve 434 that may operate to allow or prevent the flow of fluid from a patient catheter into filter device 450.

In various embodiments, catheter valve 434 may be or may include a one-way stopper or door 436 configured to allow the flow of fluid into filter device 450 via inlet port 410 and to prevent the flow of fluid in the opposite direction, namely, out of filter device 450 via inlet port 410. For example, if fluid flows in a direction from inside filter device 450 in a direction toward inlet port 410, the fluid may contact stopper 436, causing it to rise and close inlet port 410 (see, for example, FIG. 4B).

Filter device 450 may include an outlet port 412 coupled to outlet tubing 422. In various embodiments, filter device 450 may include a collection port 414 operably coupled to a collection valve 432 and in fluid communication with collection tubing 424.

FIG. 4A depicts filter device 450 in a drain configuration in which catheter valve 434 is open and collection valve 432 is closed. In some embodiments, collection valve 432 may include a shelf, sealing surface, or other structure 466 that may be used to open/close collection valve 432. For example, in the configuration depicted in FIG. 4A, sealing surface 466 may be arranged to block, seal, or otherwise close opening 464 so that fluid may not flow out of filter device 450 through collection port 414. Accordingly, PD effluent may flow into filter device 450 via inlet port 410 and through filter 430 and out of filter device 450 via outlet port 412.

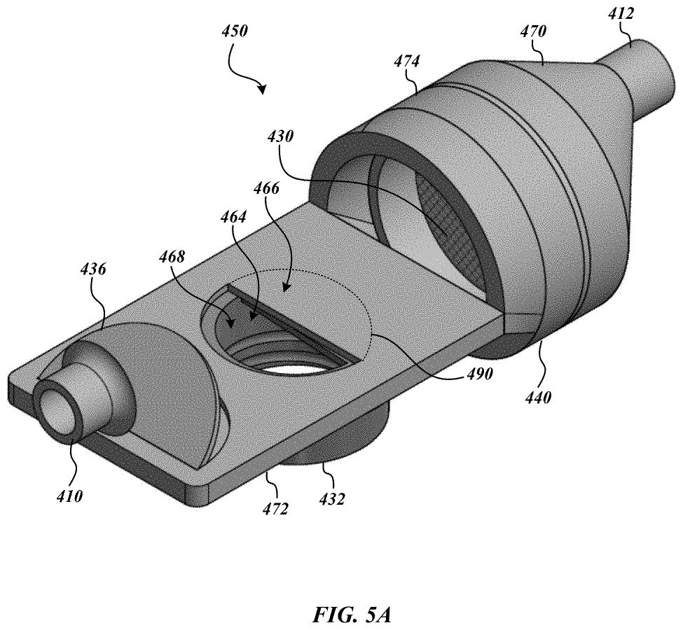

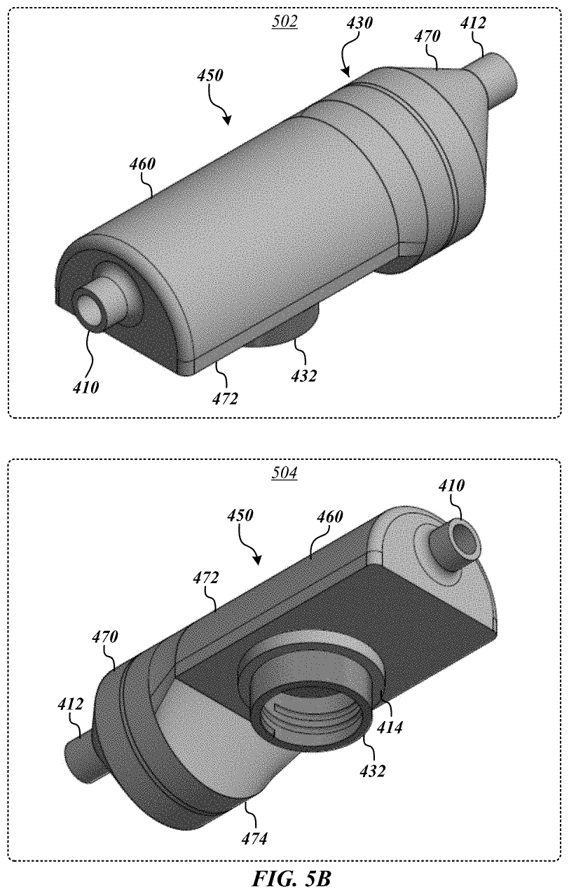

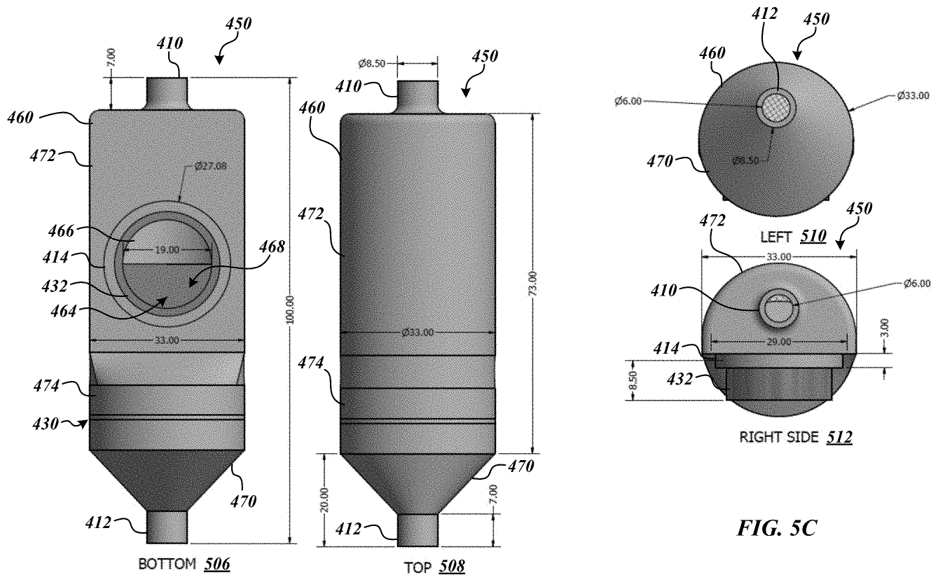

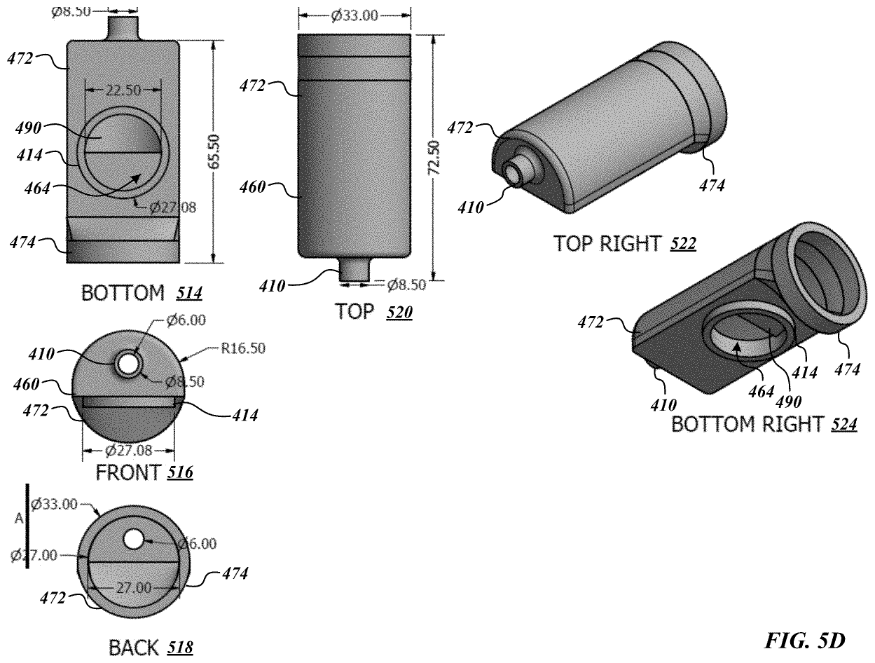

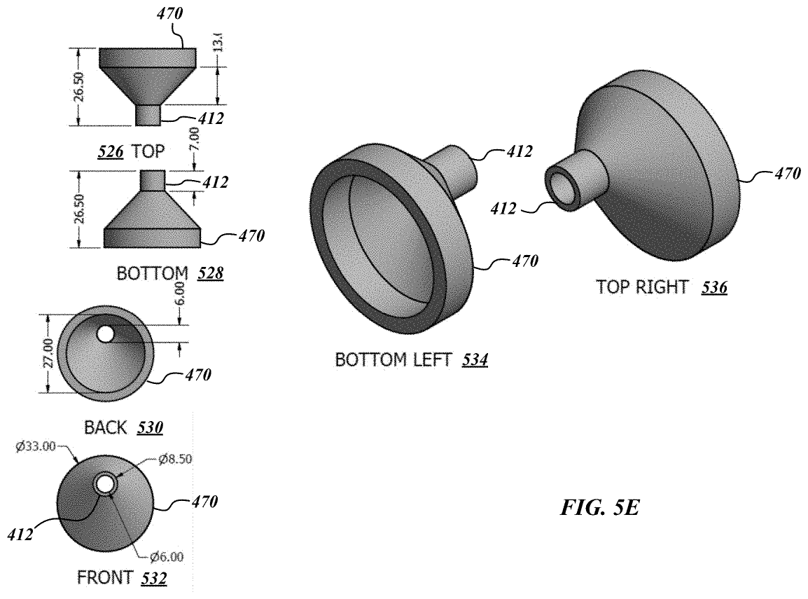

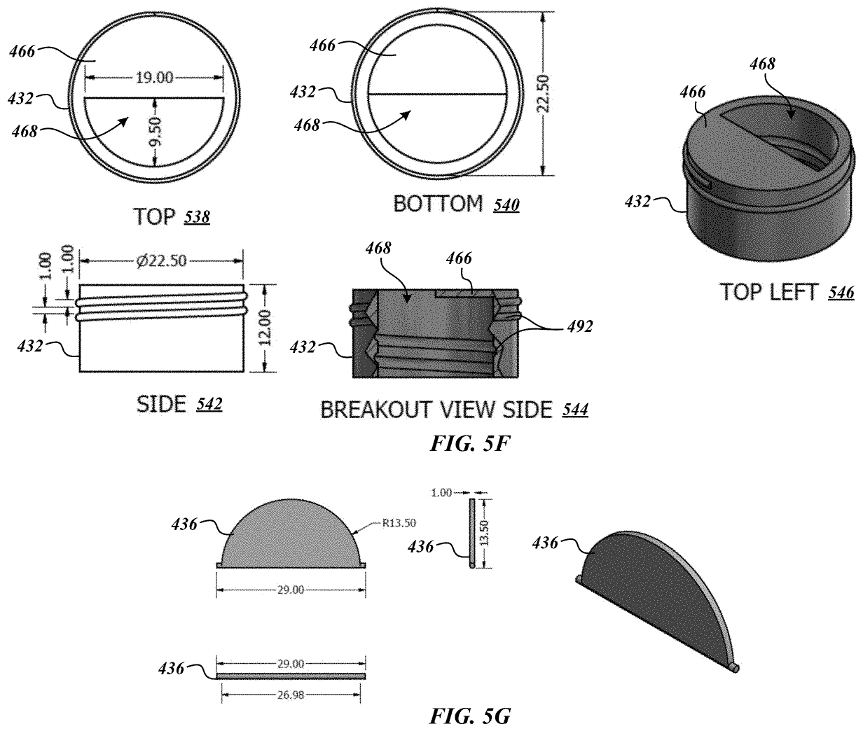

FIG. 4B illustrates collection-valve filter device 450 in a collection configuration according to some embodiments. As shown in FIG. 4B, in a collection configuration, collection valve 432 is open and stopper 436 is in a position that blocks fluid from flowing out of inlet port 410. For example, sealing surface 466 may be moved out of the way of opening 468. Accordingly, as a patient flows fluid into filter device 450 via outlet port 412, the fluid may flow through opening 464 of filter device 450, opening 468 of collection valve 432 (see, for example, FIG. 5A), and collection port 414 to a collection tube 424 or other collection device. As the fluid flows into filter device via outlet port 412, through filter 430, residue collected via filter 430 may be rinsed out of filter device 450 and into collection tube 424.

FIGS. 5A-5G depict various views of components of a collection-valve filter device according to some embodiments. FIG. 5A depicts a perspective view of a collection-valve filter device without illustrating the housing. Filter device 450 may include a first portion 472 and a second portion 470. First portion 472 may form cavity 462 with housing (not shown, see FIG. 4A). Filter 430 may be installed in an upper region or rim 474 of first portion 472 coupled to second portion 470. In some embodiments, second portion 470 may be removed to access filter 430. In various embodiments, first portion 472 and second portion 470 may be affixed to each other, for instance, via corresponding threads, a friction fit, flanges, and/or the like. First portion 472 and second portion 470 may operate to hold filter 430 rigidly in place within filter device 450. First portion 472 and second portion 470 may form a filter body or unit 440 for filter device 450.

In various embodiments, first portion 472 may include a shelf, surface, overhang, and/or the like 490 (outlined by the dashed line; see, for example, FIG. 5D). In some embodiments, collection valve 432 may include a shelf or top surface 466 (for example, in the configuration depicted in FIG. 5A, arranged under surface 490; see, for example, FIG. 5G). In a closed position, for example, when filter device 450 is in a drain configuration, surface 466 may be arranged to close or block opening 468 (for instance, moved from under surface 490 to align with opening 468). In an open configuration, for example, when filter device 450 is in a collection configuration, surface 466 may be arranged under surface 490 such that opening 468 of collection valve 432 lines up with opening 466 of first portion 472 to form a pathway for fluid to flow out of filter device 450 via collection valve 432. In some embodiments, a user may rotate collection valve 432 from the open state to the closed state. In other embodiments, an actuator or other device may operate to place collection valve 432 in the open state or the closed state, for example, by rotating, retracting, or otherwise moving surface 466 to line up opening 464 and opening 468.

FIG. 5B depicts perspective top 502 and bottom 504 views of filter device 450. FIG. 5C depicts bottom 506, top 508, left 510, and right 512 views of filter device 450. FIG. 5D depicts bottom 514, front 516, back 518, top 520, top right 522, and bottom right 524 perspective views of first portion 472. FIG. 5E depicts top 526, bottom 528, back 530, front 532, bottom left 534, and top right 536 views of second portion 470. FIG. 5F depicts top 538, bottom 540, side 542, breakout side 544, and top left 546 views of collection valve 432. As shown in FIG. 5F, collection valve 432 may include various threads 492 for example, to allow coupling with and/or rotation about collection valve 414 or other portions of filter device 450. FIG. 5G depicts various perspective views of stopper 436. The dimensions provided in FIGS. 5C-5G are in millimeters and are for non-restrictive, illustrative purposes only. Views 534, 536, and 546 are depicted in a 2:1 scale. Filter devices according to some embodiments may be sized to different dimensions.

Included herein are one or more flow diagrams representative of exemplary methodologies for performing novel aspects of the present disclosure. While, for purposes of simplicity of explanation, the one or more methodologies shown herein are shown and described as a series of acts, those skilled in the art will understand and appreciate that the methodologies are not limited by the order of acts. Some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation. Blocks designated with dotted lines may be optional blocks of a logic flow.

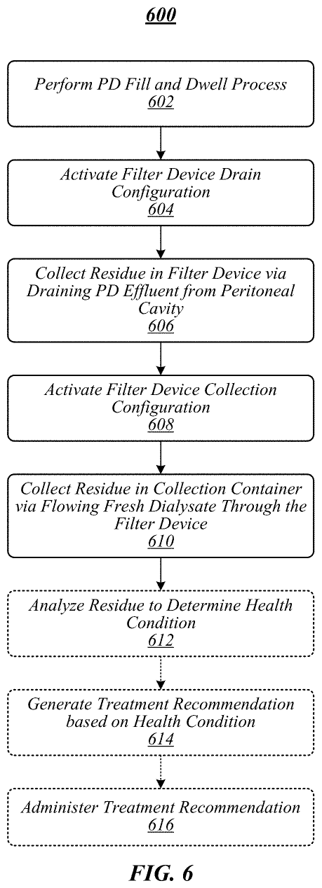

FIG. 6 illustrates a method flow for collecting PD effluent material using a collection-valve filter device according to some embodiments. At block 602, method flow 600 may include performing PD fill and dwell processes. For example, fresh dialysate from a dialysate bag or other source may be infused into a peritoneal cavity of a patient via a catheter. The dialysate may remain or “dwell” in the peritoneal cavity for a predetermined dwell time. Method flow 600 may include activating a drain configuration for a filter device at block 604. For example, in reference to FIG. 4A, during a drain stage of a PD process, collection valve 432 may be placed in a closed position blocking the flow of fluid through outlet port 414 and stopper 436 may be in an open position allowing the flow of PD effluent into filter via inlet port 410.

At block 606, method flow 600 may include collecting residue in filter device via draining PD effluent from the peritoneal cavity of a patient. For example, as PD effluent is drained from the peritoneal cavity of a patient, the PD effluent may flow through inlet port 410 of filter device 450 and through filter 430. The filtrate may exit filter device 450 through outlet port 412, and the residue (for example, cells, microorganisms, and/or other materials) may remain within filter device 450.

Method flow 600 may include activating a device collection configuration of a filter device at block 608. For example, after the drain stage of the PD process is complete, collection valve 432 may be opened and the patient may configure the PD tubing to allow fresh dialysate (or other fluid medium) to flow into filter device 450 via outlet port 412. Stopper 436 may move into a closed position to prevent the flow of fluid out of filter device 450 via inlet port 410. At block 610, method flow 600 may include collecting the residue in a collection container via flowing fresh dialysate through the filter device. For example, a patient may cause the flow of fresh dialysate through outlet port 412 to collect at least a portion of the residue on filter 430 and elsewhere within filter device 450 and cause the residue to flow out of filter device 450 via collection valve 432 and collection port 414, for example, into a collection tube 424 or other container.

At block 612, method flow 600 may include analyzing the residue to determine a health condition. For example, the residue solution collected in collection tube 424 may be stored (for instance, at 2-8° C.) before being sent to a laboratory for diagnosis (and/or banking services). During diagnosis, a laboratory process may determine a health condition of the patient, such as an infection status, a peritoneal membrane status, a morbidity classification, presence of stem cells, and/or the like. Method flow 600 may include generating a treatment recommendation based on the health condition at block 614. For example, a healthcare professional and/or automated healthcare system may generate a treatment recommendation based on the health condition determined based on the PD effluent residue analysis. For instance, the treatment recommendation may include changing a PD prescription, a treatment regimen for an infection, recommendation to bank stem cells, and/or the like. Method flow 600 may include administering the treatment recommendation at block 616. For example, the treatment recommendation may be provided to the patient and/or a healthcare professional (i.e., a physician treating the patient). The patient and/or healthcare professional may administer the treatment recommendation to treat a condition of the patient, such as renal disease, infection, inflammation, peritoneal membrane degradation, and/or the like, or to provide a service such as banking available stem cells. Embodiments are not limited in this context.

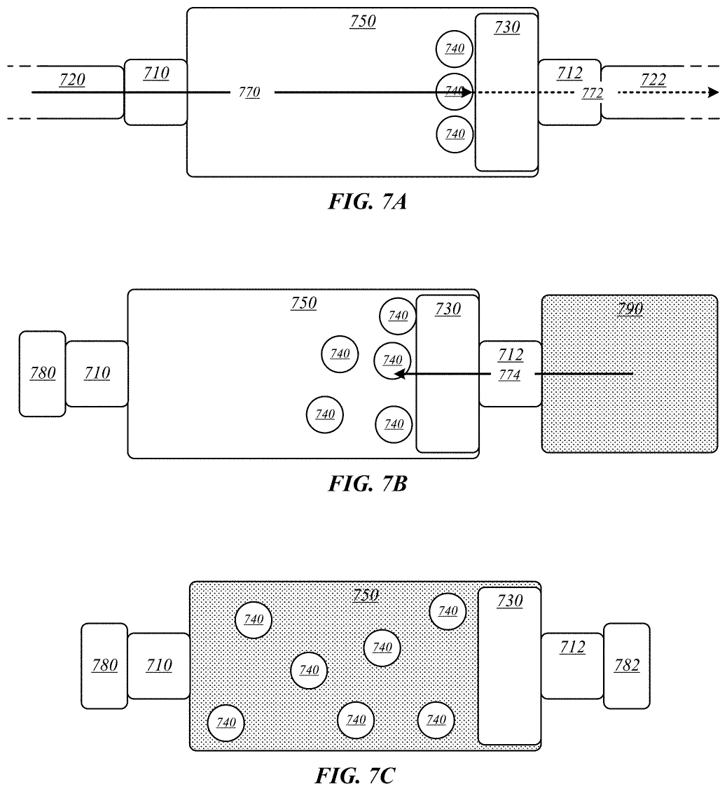

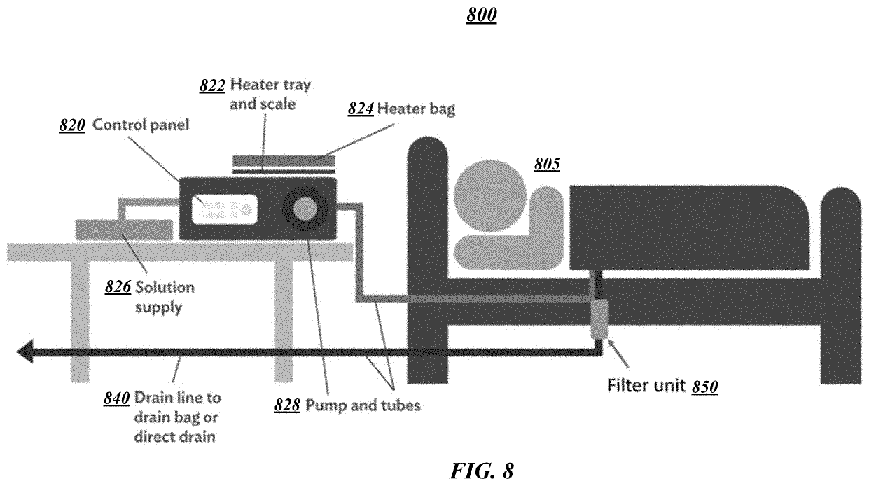

FIG. 7A illustrates a container filter device in a drain configuration according to some embodiments. FIG. 7B illustrates the container filter device of FIG. 7A in a preservation configuration according to some embodiments and FIG. 7C illustrates the container filter device of FIG. 7A in a container configuration according to some embodiments.

As shown in FIG. 7A, a collection-valve filter device 750 may include an inlet port 710 coupled to tubing 720 in fluid communication with a patient catheter. A filter 730 may be arranged within filter device 750 in fluid communication with outlet port 712 coupled to tubing 722. In the drain configuration, PD effluent 770 may flow from a patient catheter, through tubing 720, and into filter device 750 via inlet port 710. PD effluent 770 may be filtered via filter 730, leaving behind residue 740 (for instance, cells, microorganisms, and/or other materials) and allowing filtrate 772 to flow out of filter device 750 via outlet port 712 and to drain through drain tubing 722.

Referring to FIG. 7B, once the draining stage of a PD process is complete, inlet port 710 may be closed, for example, via removing (or clamping) tubing 720 and/or coupling a cap 780 or other structure to inlet port 710. A fluid container or bag 790 may be fluidically coupled to filter device 750, for example, via outlet port 712. The fluid contents 774 of fluid container 790 may flow into filter device 750. In some embodiments, fluid contents 774 may include fresh dialysate, water, and/or a preservation fluid (for instance, a medium to preserve cells, microorganisms, chemicals, biological structures, proteins, and/or the like).

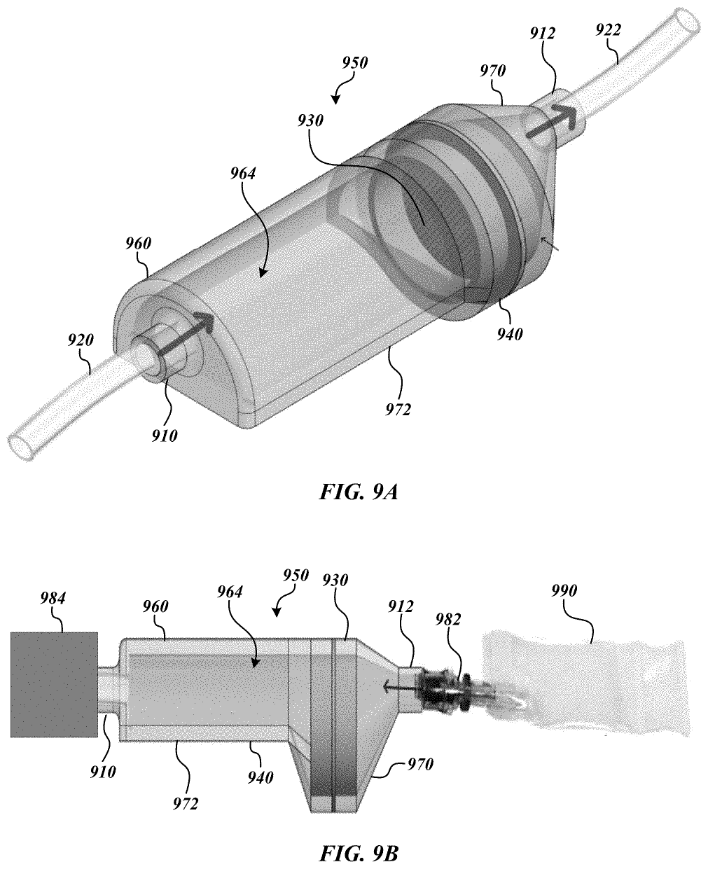

As shown in FIG. 7C, once a sufficient volume of fluid contents 774 of fluid container 790 have entered filter device 750, the outlet end of filter device 750 may be closed, sealed, or otherwise blocked off. In some embodiments, a sealing element 782 may be coupled to filter device 750, for instance, via outlet port 712 to close filter device 750. In various embodiments, sealing element 782 may include a cap or other structure. In other embodiments, sealing element 782 may be fluid container 790. For example, fluid container 790 may be a bag or pouch that may be coupled to filter device 750 (see, for example, FIG. 9B). Once the contents of the bag or pouch have been emptied or otherwise transferred, at least in part, into filter device 750, the bag or pouch may remain on filter device 750 to seal filter device. In the container configuration depicted in FIG. 7C, filter device 750 is essentially a storage container for residue 740 (or a solution containing residue 740).

FIG. 8 depicts an APD (or CCPD) configuration according to some embodiments. As shown in FIG. 8, operating environment 800 may include an APD configuration for a patient 805 to perform APD. A PD system may include a solution supply 826, control panel 820, heater tray and scale 822, and heater bag 824. A pump and tubes 828 may operate to infuse patient 805 with dialysate and to continuously flow PD effluent out of patient 805 through drain line 840. In various embodiments, a filter unit 850 may be arranged in drain line 840. In various embodiments, filter unit 850 may include a container filter device that may be more conducive for APD. Accordingly, during the APD process, PD effluent may be continuously filtered via filter unit 850 and the filtrate flowed to drain via drain line 840.

FIG. 9A illustrates a container filter device in a drain configuration according to some embodiments. FIG. 9B illustrates the container filter device of FIG. 9A in a preservation configuration according to some embodiments. As shown in FIG. 9A, a filter device 950 may include a housing 960 configured to form a cavity 964 within filter device 950. Filter device 950 may include a first portion 972 and a second portion 970. A filter 930 may be arranged between first portion 972 and second portion 970. In some embodiments, second portion 970 may be removed to access filter 930. In various embodiments, first portion 972 and second portion 970 may be affixed to each other, for instance, via corresponding threads, a friction fit, flanges, and/or the like. First portion 972 and second portion 970 may operate to hold filter 930 rigidly in place within filter device 950. First portion 972 and second portion 970 may form a filter body or unit 940 for filter device 950.

Tubing 920 may be in fluid communication with filter device via inlet port 910. PD effluent may flow from the patient to filter device 950 via tubing 920. In the drain configuration, for instance, during the drain stage of a PD process, the PD effluent may flow through cavity 964 and filter 930, then out of filter device 950 via outlet port 912. Tubing 922 may be used as a conduit for the filtrate resulting from filtering the PD effluent via filter 930 to be carried to a drain component for storing or disposing of the spent dialysate.

In FIG. 9B, filter device 950 has been activated in a collection configuration, for example, after the PD process has completed. In some embodiments, tubing 920 to the catheter may be removed and/or capped. For example, a (liquid proof) cap 984 with air releasing valve may be coupled to filter device 950, for instance, at inlet port 910 to seal the inlet end of filter device 950. A fluid pouch 990 or other fluid-filled container may be fluidically coupled to outlet port 912, for instance, via a threaded or friction-fit port 982. Fluid from within fluid pouch 990 may be forced into filter device 950 via outlet port 912. In this manner, residue filtered via filter 930 may be stored within filter device 950 and/or washed off of filter 930. In some embodiments, fluid pouch 990 may be removed and a cap or other structure (not shown) may be coupled to the outlet end of filter device 950, for instance, at outlet port 912, to seal the outlet end of filter device 950. In various embodiments, fluid pouch 990 may remain coupled to filter device 950 and operate to seal filter device 950. Accordingly, in some embodiments, filter device 950 may operate as a filtering unit and a storage or container unit for filtered residue and/or storage or preservation fluids.

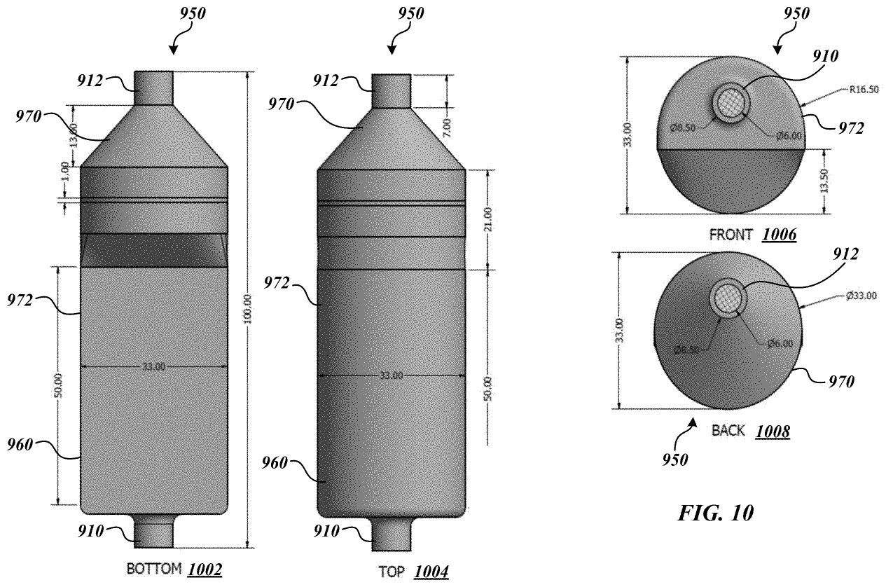

FIG. 10 depicts various views of a container filter device according to some embodiments. More specifically, FIG. 10 depicts bottom 1002, top 1004, front 1006, and back 1008 perspective views of container filter device 950 according to some embodiments. The dimensions provided in FIG. 10 are in millimeters and are for non-restrictive, illustrative purposes only. Filter devices according to some embodiments may be sized to different dimensions.

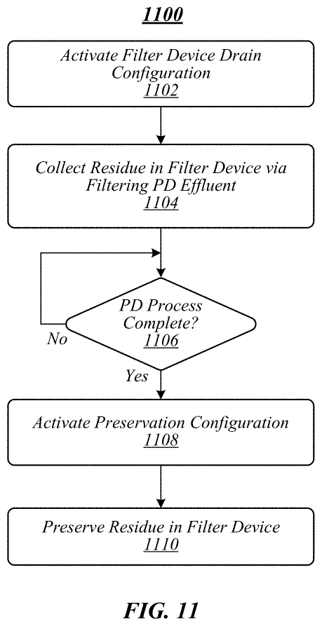

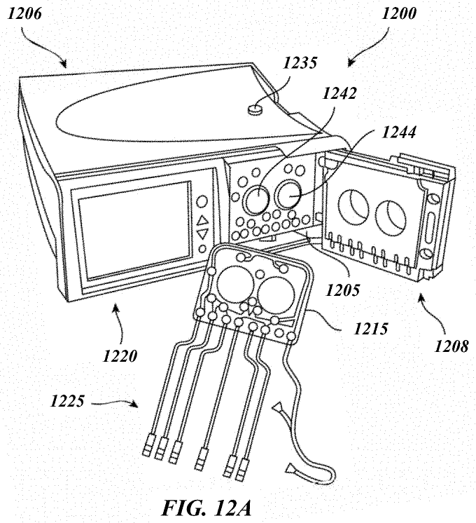

FIG. 11 illustrates a method flow for collecting PD effluent material using a container filter device according to some embodiments. At block 1102, method flow 1100 may include activating a filter device in a drain configuration. For example, in some embodiments a filter device may be connected to a PD drain line (for example, filter device 850 connected to drain line 840 in FIG. 8). In some embodiments, a container filter device may be provided as part of a tubing alternation kit provided to patients. In various embodiments, the tubing alternation kit may include a tubing cutter, tubing adapters to connect the device and drain bag, disinfection wipes and/or an extension tubing to toilet, and/or other components to allow a patient to easily and effectively couple a container filter device within the drain line. During an operating stage of an APD process, the container filter unit may be in a drain configuration (see, for example, filter device 950 of FIG. 9A).

Method flow 1100 may include collecting residue in the filter device via filtering PD effluent at block 1104. For example, once dialysis starts, a PD cycler may perform the infusion and draining of dialysate fluid. All the spent fluid (for instance, PD effluent) may go through a container filter device before entering the draining container (for instance, for the spent dialysate or PD effluent filtrate). In this manner, cells, microorganisms, and other materials in PD effluent large enough to be captured by the pore size of the filter device may be captured as PD effluent residue.

When the PD process is complete, as determined at block 1106 of method flow 1100, block 1108 of method flow 1100 may include activation of the preservation configuration of the filter device. For example, when patient finishes their treatment, they may detach the catheter end of the device (for instance, inlet port 910) and cap the device with a sterilized cap (for instance, cap 984). A pouch of cell or microorganism preservation fluid (such as Dulbecco's Modified Eagle Medium for cells, nutrient broths for bacteria, and/or other materials depending on the residue materials of interest) may be connected to the drain bag end of the filter (for instance, outlet port 912). At block 1110, method flow 1000 may include preserving the residue filtered in the filter device. For example, the preserving fluid in the pouch may be squeezed into the filter device. Once all or a sufficient amount of the preserving fluid has flowed into the filter device, the drain end of the filter device may be capped and the filter device has become a sealed, self-contained container storing the filter residue. Accordingly, in some embodiments, the entire container filter device may be shipped to laboratories or used, for example, in a point-of-care device for instance, for diagnostic and/or treatment purposes the same or similar as described in method blocks 612-616 of FIG. 6.

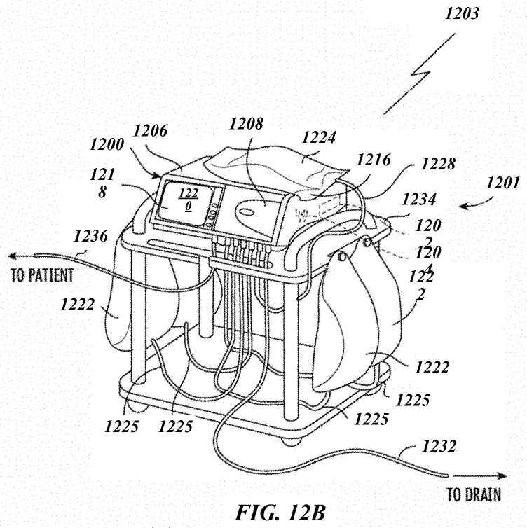

FIGS. 12A and 12B show an example of a peritoneal dialysis (PD) system 1201, which is configured in accordance with an exemplary embodiment of the system described herein. In some implementations, the PD system 1201 may be a home PD system, e.g., a PD system configured for use at a patient's home. The dialysis system 1201 may include a dialysis machine 1200 (e.g., a peritoneal dialysis machine 1200, also referred to as a PD cycler) and in some embodiments the machine may be seated on a cart 1234.

The dialysis machine 1200 may include a housing 1206, a door 1208, and a cartridge interface including pump heads 1242, 1244 for contacting a disposable cassette, or cartridge 1215, where the cartridge 1215 is located within a compartment formed between the cartridge interface and the closed door 1208 (e.g., cavity 1205). Fluid lines 1225 may be coupled to the cartridge 1215 in a known manner, such as via a connector, and may further include valves for controlling fluid flow to and from fluid bags including fresh dialysate and warming fluid. In another embodiment, at least a portion of the fluid lines 1225 may be integral to the cartridge 1215. Prior to operation, a user may open the door 1208 to insert a fresh cartridge 1215, and to remove the used cartridge 1215 after operation.

The cartridge 1215 may be placed in the cavity 1205 of the machine 1200 for operation. During operation, dialysate fluid may be flowed into a patient's abdomen via the cartridge 1215, and spent dialysate, waste, and/or excess fluid may be removed from the patient's abdomen via the cartridge 1215. The door 1208 may be securely closed to the machine 1200. Peritoneal dialysis for a patient may include a total treatment of approximately 10 to 30 liters of fluid, where approximately 2 liters of dialysate fluid are pumped into a patient's abdomen, held for a period of time, e.g., about an hour, and then pumped out of the patient. This is repeated until the full treatment volume is achieved, and usually occurs overnight while a patient sleeps.

A heater tray 1216 may be positioned on top of the housing 1206. The heater tray 1216 may be any size and shape to accommodate a bag of dialysate (e.g., a 5 L bag of dialysate) for batch heating. The dialysis machine 1200 may also include a user interface such as a touch screen 1218 and control panel 1220 operable by a user (e.g., a caregiver or a patient) to allow, for example, set up, initiation, and/or termination of a dialysis treatment. In some embodiments, the heater tray 1216 may include a heating element 1235, for heating the dialysate prior to delivery into the patient.

Dialysate bags 1222 may be suspended from hooks on the sides of the cart 1234, and a heater bag 1224 may be positioned in the heater tray 1216. Hanging the dialysate bags 1222 may improve air management as air content may be disposed by gravity to a top portion of the dialysate bag 1222. Although four dialysate bags 1222 are illustrated in FIG. 12B, any number “n” of dialysate bags may be connectable to the dialysis machine 1200 (e.g., 1 to 5 bags, or more), and reference made to first and second bags is not limiting to the total number of bags used in a dialysis system 1201. For example, the dialysis machine may have dialysate bags 1222a, . . . 1222n connectable in the system 1201. In some embodiments, connectors and tubing ports may connect the dialysate bags 1222 and lines for transferring dialysate. Dialysate from the dialysate bags 1222 may be transferred to the heater bag 1224 in batches. For example, a batch of dialysate may be transferred from the dialysate bags 1222 to the heater bag 1224, where the dialysate is heated by the heating element 1235. When the batch of dialysate has reached a predetermined temperature (e.g., approximately 98°−100° F., 37° C.), the batch of dialysate may be flowed into the patient. The dialysate bags 1222 and the heater bag 1224 may be connected to the cartridge 1215 via dialysate bag lines or tubing 1225 and a heater bag line or tubing 1228, respectively. The dialysate bag lines 1225 may be used to pass dialysate from dialysate bags 1222 to the cartridge during use, and the heater bag line 1228 may be used to pass dialysate back and forth between the cartridge and the heater bag 1224 during use. In addition, a patient line 1236 and a drain line 1232 may be connected to the cartridge 1215. The patient line 1236 may be connected to a patient's abdomen via a catheter and may be used to pass dialysate back and forth between the cartridge and the patient's peritoneal cavity by the pump heads 1242, 1244 during use. The drain line 1232 may be connected to a drain or drain receptacle and may be used to pass dialysate from the cartridge to the drain or drain receptacle during use.

Although in some embodiments, dialysate may be batch heated as described above, in other embodiments, dialysis machines may heat dialysate by in-line heating, e.g., continuously flowing dialysate through a warmer pouch positioned between heating elements prior to delivery into a patient. For example, instead of a heater bag for batch heating being positioned on a heater tray, one or more heating elements may be disposed internal to the dialysis machine. A warmer pouch may be insertable into the dialysis machine via an opening. It is also understood that the warmer pouch may be connectable to the dialysis machine via tubing (e.g., tubing 1225), or fluid lines, via a cartridge. The tubing may be connectable so that dialysate may flow from the dialysate bags, through the warmer pouch for heating, and to the patient.

In such in-line heating embodiments, a warmer pouch may be configured so dialysate may continually flow through the warmer pouch (instead of transferred in batches for batch heating) to achieve a predetermined temperature before flowing into the patient. For example, in some embodiments the dialysate may continually flow through the warmer pouch at a rate between approximately 100-300 mL/min. Internal heating elements (not shown) may be positioned above and/or below the opening, so that when the warmer pouch is inserted into the opening, the one or more heating elements may affect the temperature of dialysate flowing through the warmer pouch. In some embodiments, the internal warmer pouch may instead be a portion of tubing in the system that is passed by, around, or otherwise configured with respect to, a heating element(s).

The touch screen 1218 and the control panel 1220 may allow an operator to input various treatment parameters to the dialysis machine 1200 and to otherwise control the dialysis machine 1200. In addition, the touch screen 1218 may serve as a display. The touch screen 1218 may function to provide information to the patient and the operator of the dialysis system 1201. For example, the touch screen 1218 may display information related to a dialysis treatment to be applied to the patient, including information related to a prescription.

The dialysis machine 1200 may include a processing module 1202 that resides inside the dialysis machine 1200, the processing module 1202 being configured to communicate with the touch screen 1218 and the control panel 1220. The processing module 1202 may be configured to receive data from the touch screen 1218 the control panel 1220 and sensors, e.g., weight, air, flow, temperature, and/or pressure sensors, and control the dialysis machine 1200 based on the received data. For example, the processing module 1202 may adjust the operating parameters of the dialysis machine 1200.

The dialysis machine 1200 may be configured to connect to a network 1203. The connection to network 1203 may be via a wired and/or wireless connection. The dialysis machine 1200 may include a connection component 1204 configured to facilitate the connection to the network 1203. The connection component 1204 may be a transceiver for wireless connections and/or other signal processor for processing signals transmitted and received over a wired connection. Other medical devices (e.g., other dialysis machines) or components may be configured to connect to the network 1203 and communicate with the dialysis machine 1200.

The user interface portion such as the touch screen 1218 and/or control panel 1220 may include one or more buttons for selecting and/or entering user information. The touch screen 1218 and/or control panel 1220 may be operatively connected to a controller (not shown) and disposed in the machine 1200 for receiving and processing the inputs to operate the dialysis machine 1200.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A collection-valve filter device for filtering dialysis effluent, comprising:
a filter body comprising an inlet port, an outlet port, and a collection port, the inlet port and the outlet port arranged at opposite ends of the filter body, the collection port arranged in a bottom portion of the filter body, the inlet port configured to receive the dialysis effluent draining from a catheter fluidically coupled to a patient;
a catheter valve configured to move to one of an inlet closed position to close the inlet port or an inlet open position to open the inlet port; and
a collection valve operably coupled to the collection port, the collection valve to move to a collection open position to open the collection port and a collection closed position to close the collection port.

2. The collection-valve filter device of claim 1, the collection-valve filter device being in a drain configuration responsive to the catheter valve being in the inlet open position and the collection valve being in the collection closed position.

3. The collection-valve filter device of claim 1, the collection-valve filter device is in a collection configuration responsive to the catheter valve being in the inlet closed position and the collection valve being in the collection open position.

4. The collection-valve filter device of claim 1, the collection-valve filter device operative to allow a flow of the dialysis effluent through the inlet port and the filter, and filtrate out through the outlet port, in the drain configuration.

5. The collection-valve filter device of claim 1, the collection-valve filter device operative to allow a flow of fresh dialysate through the outlet port, the filter, and out through the collection port in the collection configuration.

6. The collection-valve filter device of claim 1, the filter to capture materials in the dialysis effluent, the materials comprising at least one of patient cells or microorganisms.

7. The collection-valve filter device of claim 1, the collection-valve filter configured to filter peritoneal dialysis (PD) effluent during a PD treatment.

8. The collection-valve filter device of claim 1, the collection-valve filter configured to filter PD effluent during continuous ambulatory peritoneal dialysis (CAPD) treatment.

9. The collection-valve filter device of claim 1, the collection-valve filter device configured to be installed in drain tubing of a CAPD system.

10. A method of treating a dialysis patient, comprising:
collecting a peritoneal dialysis (PD) effluent residue during a PD treatment of the patient using a collection-valve filter device operatively coupled to a PD system, the collection-valve filter device comprising:
a filter body;
an inlet port;
an outlet port; and
a filter arranged between the inlet port and the outlet port to filter the dialysis effluent and collect the PD effluent residue;
analyzing the PD effluent residue to determine a health condition of the dialysis patient; and
determining a treatment recommendation based on the health condition.

11. The method of claim 10, the treatment recommendation comprising harvesting stem cells from the PD effluent residue.

12. The method of claim 10, comprising administering the treatment recommendation.

13. The method of claim 10, comprising placing the collection-valve filter device in one of:
a drain configuration wherein the inlet port is operative to receive the dialysis effluent from a drain line of a dialysis system; or
a preservation configuration wherein the inlet port is closed and a fluid container is fluidically coupled to the outlet port.

14. A container filter device for filtering dialysis effluent, comprising:
a filter body having an inlet port and an outlet port arranged at opposite ends thereof; and
a filter arranged between the inlet port and the outlet port,
wherein, in a drain configuration, the inlet port is operative to receive the dialysis effluent from a drain line of a dialysis system, the dialysis effluent to flow through the filter to capture residue in the dialysis effluent and generate filtrate to flow out of the container filter device via the outlet port.

15. The container filter device of claim 14, wherein, in a preservation configuration, the inlet port is closed and a fluid container is fluidically coupled to the outlet port.

16. The container filter device of claim 15, wherein, in the preservation configuration, the outlet port is configured to receive fluid arranged within the fluid container, the fluid to flow through the filter into the filter body.

17. The container filter device of claim 14, the filter to capture materials in the dialysis effluent, the materials comprising at least one of patient cells or microorganisms.

18. The container filter device of claim 14, the collection-valve filter configured to filter peritoneal dialysis (PD) effluent during a PD treatment.

19. The container filter device of claim 14, the collection-valve filter configured to filter PD effluent during automated peritoneal dialysis (APD) or continuous cyclic peritoneal dialysis (CCPD) treatment.

20. The container filter device of claim 14, the collection-valve filter device configured to be installed in drain tubing of an APD or a CCPD system.
